(19) United States
(12) Patent Application Publication
(10) Pub. No.: US 2024/0307092 A1
(43) Pub. Date: Sep. 19, 2024

(54) INSERTION DEVICE FOR A BIOSENSOR

(71) Applicant: BIONIME CORPORATION, Taichung City (TW)

(72) Inventors: Chun-Mu Huang, Taichung City (TW); Chieh-Hsing Chen, Taichung City (TW); Kuan-Lin Chang, Taichung City (TW); Hung-Wen Chiang, Taichung City (TW)

(73) Assignee: BIONIME CORPORATION, Taichung City (TW)

(21) Appl. No.: 18/604,309

(22) Filed: Mar. 13, 2024

Related U.S. Application Data
(60) Provisional application No. 63/490,314, filed on Mar. 15, 2023.

(51) Int. Cl. A61B 17/34; A61B 5/145

(52) U.S. Cl. CPC A61B 17/3468; A61B 5/14532; A61B 2560/063

(57) ABSTRACT

An implant device for a biosensor comprises a housing unit, an implant module, a bottom seat and a sensor component. The implant module is disposed in an accommodating space of the housing unit. The implant module includes a main body unit, a guiding set, an implant seat, a first elastic member, a needle withdrawal seat, a second elastic member, and a needle implant member. When the housing unit is pressed downwardly, the implant seat is displaced downwardly to perform automatic needle implantation by virtue of an resilient force of the first elastic member; when the needle implantation is completed, a limiting relationship of the implant seat and the needle withdrawal seat is released, so the needle withdrawal seat completes automatic needle withdrawal by releasing a resilient force of the second elastic member. The implant seat and the needle withdrawal seat are guided by the guiding set to move stably, thereby achieving easy control of production tolerances, increasing production speed and yield rate, improving the stability of needle implantation, and making needle implantation more painless and sensationless.

### CROSS-REFERENCE TO RELATED APPLICATION

This application claims the benefit of U.S. Provisional Patent Application No. 63/490,314, filed on Mar. 15, 2023, which is incorporated by reference herein in its entirety.

### TECHNICAL FIELD

The present invention is related to a system and method for a biological detection system applied to an organism, and particularly to an implant device for mounting a biological detection device on a skin of the organism and implanting a biosensor under the skin of the organism.

### PRIOR ART

A conventional self-testing method for blood sugar is to draw microvascular blood from a tip of the needle, to drip the same on a blood sugar test strip, and to read a blood sugar value by a machine. As long as operation is correctly done, the obtained blood sugar value may be very precise. When the obtained blood sugar value is too high or too low, treatment may be implemented as soon as possible. It is required for some people to measure their blood sugar very often, especially for the patients who have unsatisfactory blood sugar control, who are on insulin treatment, or who have a large blood sugar fluctuations.

There is another apparatus that may realize self-testing of blood sugar, that is, continuous glucose monitoring (CGM). A detection sensor needle is placed under a skin of the patient to continuously measure a concentration of interstitial fluid glucose, since glucose in the blood diffuses into the interstitial fluid and then into cells. The sensor may estimate blood sugar levels through conversion and provide real-time blood sugar level values. The blood sugar is recorded at regular intervals and a trend curve of blood sugar variation is displayed so as to provide warnings when the blood sugar is too high or too low.

Current research shows that for patients with type 1 and type 2 diabetes requiring insulin injections, the use of CGM may reduce glycated hemoglobin by approximately 0.6% as compared to measurement of blood sugar that is measured through finger tips, and a total duration of hypoglycemia per day may be reduced.

CGM must be worn by users for a long time, so miniaturization in its volume will be an inevitable trend. The architecture of CGM includes: (a) a sensor for measuring a physiological signal corresponding to a glucose concentration in the human body; (b) a transceiver for receiving and transmitting the physiological signal, and (c) an implant device for attaching the sensor to the transceiver, attaching the transceiver to the user's skin, and implanting the sensor under the user's skin.

In order to achieve safe and accurate implantation of the sensor under the user's skin, which makes the physiological signal measured by the sensor be transmitted to a receiving instrument corresponding to the transceiver for the user to obtain a blood sugar status any time, the applicant has applied for the U.S. Patent Application Publication No. US20210030960A1 and No. US20210030344A1. However, the applicant believes that the function of the abovementioned implant instrument may be enhanced to provide faster, more stable and safer placement of the sensor under the user's skin. It is also expected that the improved sensor also facilitates easier assembly during manufacturing and production process, with production yield rate and speed being significantly improved.

### Content of Invention

Therefore, an object of the present invention is to provide an implant device for a biosensor that can improve technical issues of an existing implant device.

Accordingly, an implant device for a biosensor of the present invention comprises a housing unit, an implant module, a bottom seat and a sensor component. The housing unit has an accommodating space, the implant module is disposed in the accommodating space of the housing unit, and the implant module includes:
a main body unit linked to the housing unit, the main body unit defining a displacement space;
a guiding set connected to the main body unit and located in the displacement space;
an implant seat detachably limited to the main body unit, and guided by the guiding set to be displaceable in the displacement space;
a first elastic member having one of ends positioned relative to the main body unit, and another one of the ends elastically abutting against the implant seat;
a needle withdrawal seat detachably limited to the implant seat and guided by the guiding set;
a second elastic member elastically abutting between the implant seat and the needle withdrawal seat;
a needle implant member;
a bottom seat detachably limited to the main body unit; and
a sensor component detachably limited to the bottom seat;
wherein, the implant seat is displaced downwardly by releasing a resilient force of the first elastic member until a limiting relationship with the main body unit is released to thereby perform an automatic needle implantation; when the needle implantation is completed, a limiting relationship of the implant seat and the main body unit is released, and a limiting relationship of the implant seat and the needle withdrawal seat is released, so the needle withdrawal seat finishes an automatic needle withdrawal by releasing a resilient force of the second elastic member, and the implant seat and the needle withdrawal seat are guided by the guiding set to stably move.

The implant device for a biosensor of the present invention comprises a housing unit, an implant module, a bottom seat, and a sensor component. The housing unit includes a housing member. The implant module includes a main body unit, a guiding set, an implant seat, a first elastic member, a needle withdrawal seat, a second elastic member, and a needle implant member. When the housing member is subjected to a force to activate the needle implantation, the implant seat and the main body unit are released from a strike-limiting relationship, and the implant seat and the needle withdrawal seat are guided by the guiding set to move so the implant seat displaces downwardly to perform automatic needle implantation. After the needle implantation is completed, the implant seat and the needle withdrawal seat are released from a needle withdrawal limit relationship, such that the needle withdrawal seat displaces upwardly to complete automatic needle withdrawal, and the implant seat and the needle withdrawal seat are guided by the guiding set to stably move.

The effects of the present invention reside in the following: by utilizing the guiding set connected to the main body unit and located in the displacement space, the implant seat and the needle withdrawal seat may stably displace as being guided by the guiding set, such that achievable effects include easy control of production tolerance, increased production speed and yield rate, reduced implant needle bouncing, improved stability of needle implantation, and painless and sensationless needle implantation.

### BRIEF DESCRIPTION OF THE DRAWINGS

Other features and effects of the present invention will be apparently presented in the embodying manner with reference to the drawings, in which:

FIG. 1 is a perspective assembled sectional view of an embodiment of an implant device for a biosensor of the present invention;

FIG. 2 is a sectional view taken along line II-II in FIG. 1;

FIG. 3 is a sectional view taken along line III-III in FIG. 1, illustrating configuration of a plurality of guiding pins;

FIG. 4 is an exploded perspective view of the embodiment;

FIG. 5 is a partly exploded perspective view of the embodiment;

FIG. 6 is a perspective view of a liner member of the embodiment;

FIG. 7 is a perspective view of a main body member of the embodiment;

FIG. 8 is a schematic perspective view of an implant seat and a number of guiding pins of the embodiment;

FIG. 9 is a sectional view of the implant seat of the embodiment;

FIG. 10 is a perspective view of a needle withdrawal seat of the embodiment;

FIG. 11 is a plan view of a release layer of the embodiment;

FIG. 12 is a plan view of a release layer in another form of the embodiment;

FIG. 13 is a plan view of a release layer in still another form of the embodiment;

FIG. 14 is a schematic perspective view of a peel-off element in one of forms of the embodiment;

FIG. 15 is a schematic perspective view of a peel-off element in another one of forms of the embodiment;

FIG. 16 is a plane assembled view of the embodiment that is with a bottom cover not opened and that is in a to-be-stroke state;

FIG. 17 is a sectional view taken along line XVII-XVII in FIG. 16;

FIG. 18 is a schematic view of the embodiment that is with the bottom cover opened and that is in a needle to-be-implanting state;

FIG. 19 is a schematic operational view of the embodiment, illustrating a state where a cover is opened, a housing member is pressed, and striking is activated;

FIG. 20 is a sectional view taken along line XX-XX in FIG. 19;

FIG. 21 is a schematic operational view of a safety unlocking operation prior to striking of the embodiment;

FIG. 22 is a sectional view taken along line XII-XII in FIG. 21;

FIG. 23 is a schematic view of a needle striking operation of the embodiment;

FIG. 24 is a sectional view taken along line XXIV-XXIV in FIG. 23;

FIG. 25 is a schematic view of a needle implantation that is completed of the embodiment;

FIG. 26 is a sectional view taken along line XXVI-XXVI in FIG. 25;

FIG. 27 is a schematic view of a needle withdrawal operation of the embodiment, illustrating a state where a needle withdrawal is completed;

FIG. 28 is a sectional view taken along line XXVIII-XXVIII in FIG. 27;

FIG. 29 is a schematic view of a bottom seat and a sensor component detached from a bottom of a main body member of the embodiment;

FIG. 30 is a diagram comparing experimental results of the prior art and that of the embodiment taken by a high-speed camera; and

FIG. 31 is a schematic diagram illustrating a peeling yield rate of the release layer of the embodiment.

### EMBODYING MANNER

Before the present invention is described in detail, it should be noted that similar elements are represented by the same reference numerals in the following description.

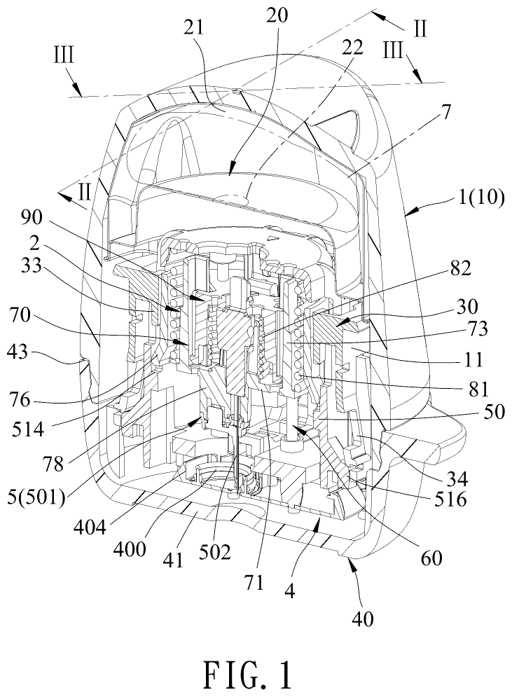

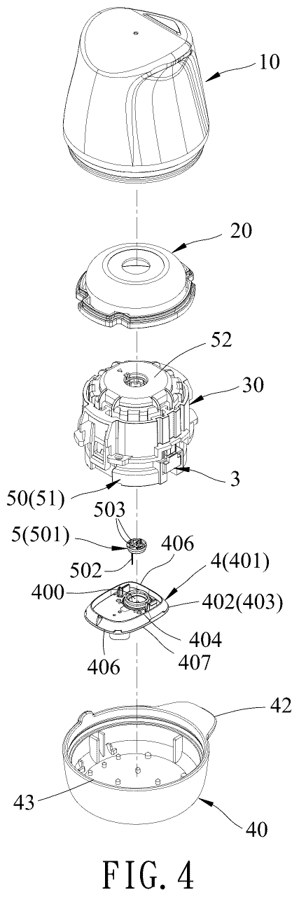

Referring to FIGS. 1 to 5, an embodiment of an implant device for a biosensor according to the present invention comprises a housing unit 1, an implant module 2, two fixing members 3, a bottom seat 4, a sensor component 5, a peel-off element 6 and a desiccant 7.

The housing unit 1 includes a housing member 10, a top cover 20 fixed in the housing member 10, an lining member 30 positioned in the housing member 10 and located at one side of the top cover 20, and a bottom cover 40 engageable with the housing member 10 in an airtight manner. The housing member 10 defines an accommodating space 11, and a chamber 21 separated from the accommodating space 11 is formed between the top cover 20 and the housing member 10. The top cover 20 may include an opening 22 that allows the desiccant 7 (in a form of a packet, tablet or granular texture) to be placed into the chamber 21 therethrough. During a product assembly process, the opening 22 is sealed after the desiccant 7 is placed into the chamber 21, but there are still orifices for preventing moist in the can. In another embodiment, if a desiccant in a form of a packet having a size greater than that of the opening 22 is used, it is not required to seal the opening 22.

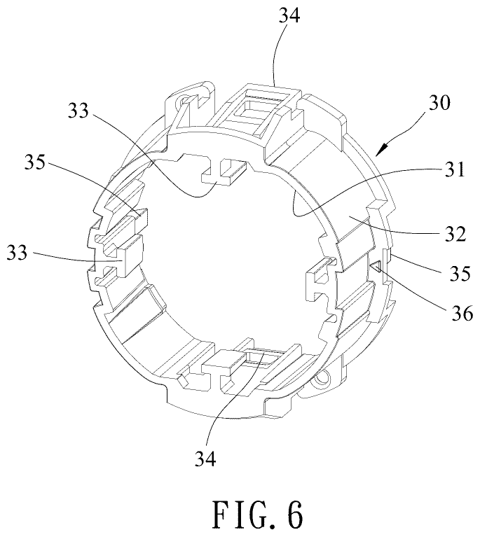

Referring to FIG. 6, the lining member 30 is a hollow annular frame, and has an inner peripheral surface 31, an outer peripheral surface 32 opposite to the inner peripheral surface 31, a number of driving portions 33 disposed on the inner peripheral surface 31, a pair of interlock portions 34 in a form of resilient plates and extending downwardly, a pair of pawls 35 extending downwardly from a bottom edge thereof, and a fourth alignment mark 36 disposed on the outer peripheral surface 32. The fourth alignment mark 36 is a triangular mark.

The bottom cover 40 is used for detachably coupling an opening of the accommodating space 11 of the housing member 10, and includes a bottom plate portion 41.

The implant module 2 is disposed in the accommodating space 11 of the housing unit 1. The implant module 2 includes a main body unit 50, a guiding set 60, an implant seat 70, a first elastic member 81, a needle withdrawal seat 90, a second elastic member 82, and a needle implant member 100.

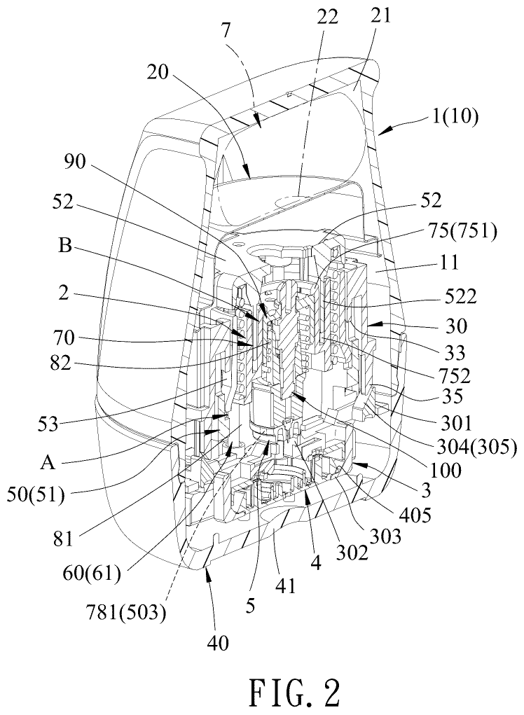

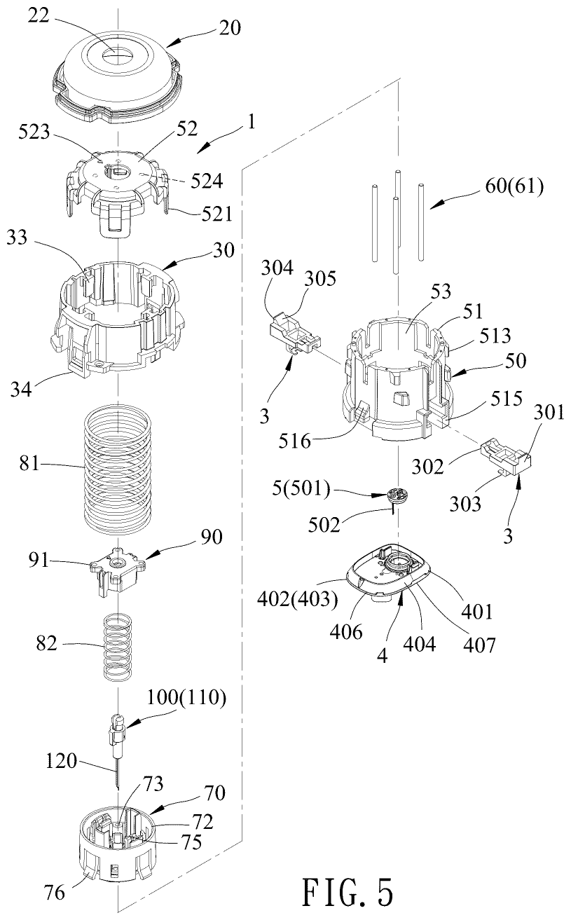

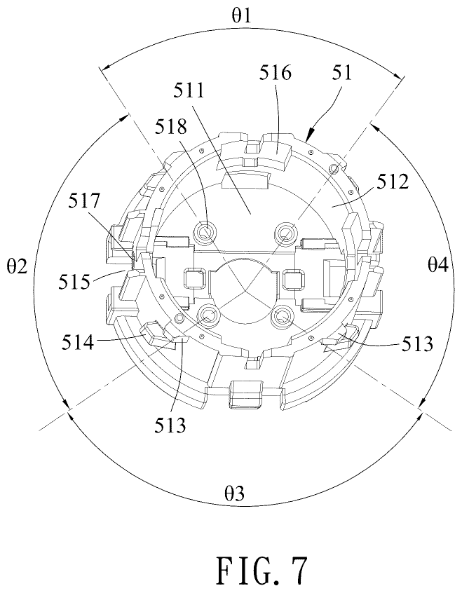

The main body unit 50 is linked to the housing unit 1 and is slidably sleeved with respect to the lining member 30. The main body unit 50 has a main body member 51 and a main body cover 52 linked detachably to the main body member 51. The main body member 51 and the main body cover 52 cooperatively compose a displacement space 53. With reference to FIG. 7, the main body member 51 has a bottom wall 511, an annular wall 512 transversely connected to the bottom wall 511, a number of engaging portions 513 disposed on the annular wall 512, a number of buckling portions 514 disposed on the annular wall 512, a pair of sliding grooves 515 disposed in the annular wall 512 and communicating with the bottom wall 511, a pair of limiting portions 516 protrudingly disposed from the annular wall 512 and respectively corresponding to the interlock portions 34, and a first alignment mark 517 disposed on the annular wall 512. The bottom wall 511 has four lower positioning holes 518, and an included angle (θ1), (θ2), (θ3), (θ4) is formed between two adjacently disposed ones of the lower positioning holes 518. The included angles (θ1), (θ2), (θ3), (θ4) are asymmetric. The first alignment mark 517 corresponds to the fourth alignment mark 36, and the first alignment mark 517 is a triangular mark. As shown in FIG. 5, the main body cover 52 has a number of connecting portions 521 engaging mutually the engaging portions 513, two limiting members 522 (see FIG. 2) plate-shaped, a second alignment mark 523 corresponding to the first alignment mark 517, and four upper positioning holes 524. The second alignment mark 523 is a triangular mark, and included angles formed between two adjacently disposed ones of the upper positioning holes 524 are respectively the same as the included angles (θ1), (θ2), (θ3), (θ4). The included angles are asymmetric.

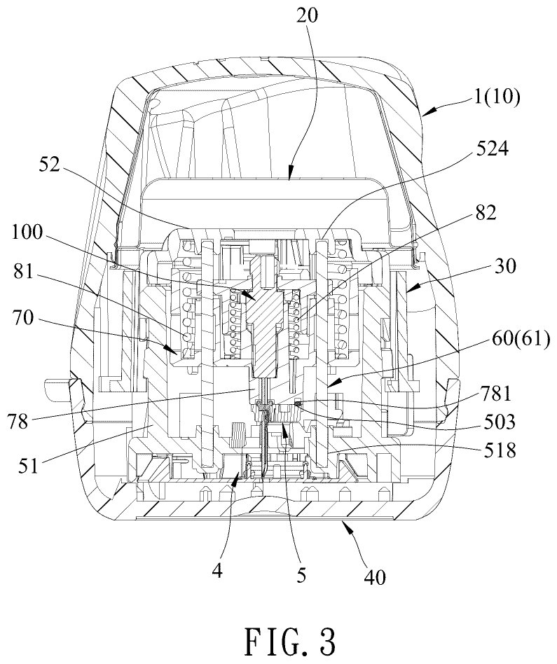

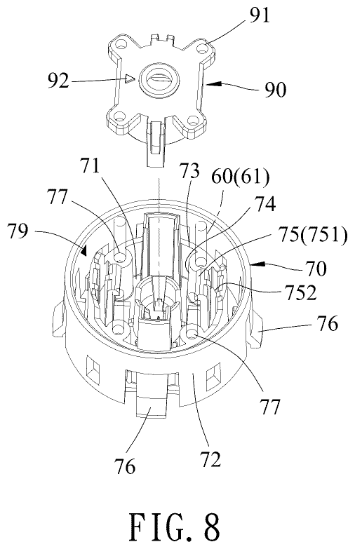

Referring to FIG. 8, the guiding set 60 is located in the displacement space 53 and has four guiding pins 61 connected between the main body member 51 and the main body cover 52. As shown in FIG. 3, one of ends of each of the guiding pins 61 is inserted into a corresponding one of the lower positioning holes 518, and another one of the ends is inserted into a corresponding one of the upper positioning holes 524. Included angles formed between two adjacently disposed ones of the guiding pins 61 are respectively the same as the included angles (θ1), (θ2), (θ3), (θ4). The included angles are asymmetric. As denoted in FIG. 7, in this embodiment, an included angle formed between two of the guiding pins 61 is not equal to an included angle formed between another two of the guiding pins 61 opposite thereto. In addition to the cylindrical shape of the guiding pins 61 as presented, cross-sectional shapes of the guiding pins may be different according to different application requirements.

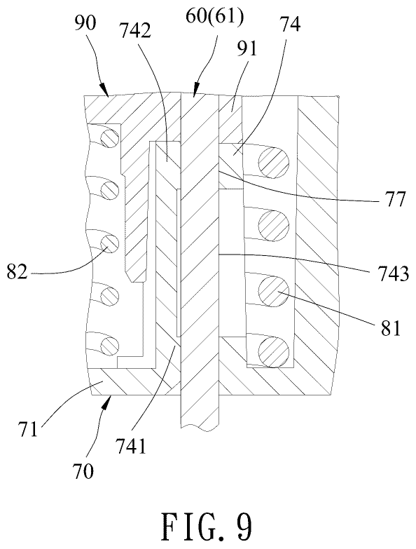

The implant seat 70 is detachably limited to the main body unit 50, and is guided by the guiding set 60 to be displaceable in the displacement space 53. The implant seat 70 has a plate member 71, an outer barrel member 72 transversely connected to the plate member 71, an inner barrel member 73 transversely connected to the plate member 71 and located in the outer barrel member 72, a number of guiding tubes 74 connected to the inner barrel member 73, a limiting component 75 connected to the inner barrel member 73 and for keeping the needle withdrawal seat 90 being positioned relative to the implant seat 70, a number of snapped-on portions 76 driven by the driving portions 33 and detachably engaging the buckling portions 514 of the main body member 51, four first guiding holes 77 provided for slidable extension of the guiding pins 61 respectively therethrough, a positioning member 78 protrudingly provided on the plate member 71, and a fifth alignment mark 79 disposed on the outer barrel member 72 and corresponding to the first alignment mark 517. The first guiding holes 77 are respectively disposed in the guiding tubes 74, and the included angles formed between two adjacently disposed ones of the first guiding holes 77 are respectively the same as the included angles (θ1), (θ2), (θ3), (θ4). The included angles are asymmetric (as shown in FIG. 8 and as denoted in FIG. 7). Referring to FIG. 9, each of the guiding tubes 74 has a bottom segment 741 connected to the plate member 71, a top segment 742 opposite to the bottom segment 741 along an axial direction of a corresponding one of the guiding pins 61, and a hollow portion 743 between the bottom segment 741 and the top segment 742 and communicating with a corresponding one of the first guiding holes 77. The limiting component 75 has a pair of buckling plates 751 having a hook shape, and a limiting groove 752 formed between the buckling plates 751 and the inner barrel member 73 is provided for insertion of the limiting members 522. The limiting component 75 limits the needle withdrawal seat 90, such that the implant seat 70, the main body cover 52, and the needle withdrawal seat 90 compose a needle withdrawal limiting structure (B). The snapped-on portions 76 are connected to the outer barrel member 72, and the snapped-on portions 76 cooperate with the buckling portions 514 such that a strike-limiting structure (A) is composed between the implant seat 70 and the main body member 51. The positioning member 78 has two linked portions 781 (see FIG. 3) that are in a form of cylindrical pillars.

One of the ends of the first elastic member 81 is positioned relative to the main body unit 50 and abuts against the main body cover 52, and another one of the ends resiliently abuts against the implant seat 70. The first elastic member 81 may be a pre-compressed spring.

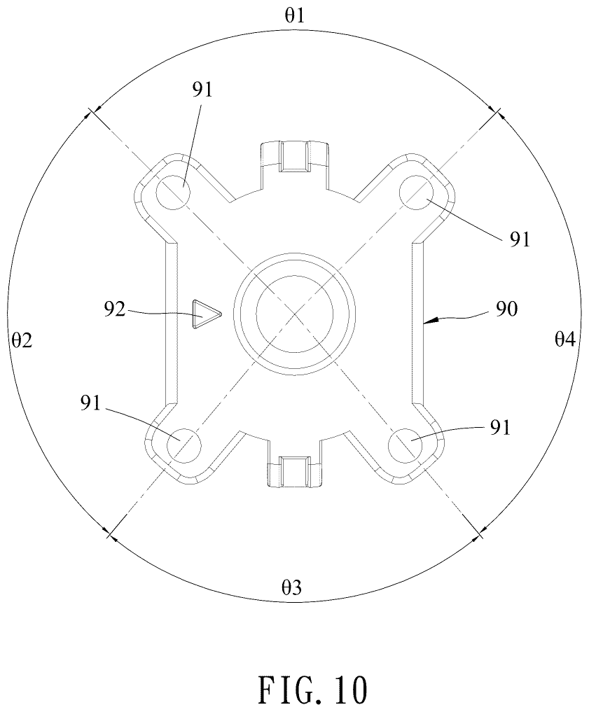

The needle withdrawal seat 90 is detachably limited to the implant seat 70 and is guided by the guiding set 60. The needle withdrawal seat 90 has four second guiding holes 91 provided for slidable extension of the guiding pins 61, respectively, and a third alignment mark 92 corresponding to the first alignment mark 517. An included angle (θ1), (θ2), (θ3), (θ4) is formed between any two adjacently disposed ones of the second guiding holes 91 and the included angles (θ1), (θ2), (θ3), (θ4) are asymmetric (see FIG. 10).

The second elastic member 82 resiliently abuts between the implant seat 70 and the needle withdrawal seat 90. The second elastic member 82 may be a pre-compressed spring.

The needle implant member 100 has a main body 110 and an implant needle 120 connected to the main body 110 and used for carrying a sensor 502.

The fixing members 3 are respectively and slidably inserted in the sliding grooves 515 of the main body member 51, and each has a pushing portion 301, a supporting portion 302 opposite to the pushing portion 301, a first engaging hook 303 located between the pushing portion 301 and the supporting portion 302, and a co-movable portion 304 located between the pushing portion 301 and the supporting portion 302. The co-movable portion 304 has a guiding driving bevel 305 that may be driven by a corresponding one of the pawls 35. In a state where the bottom cover 40 is closed relative to the housing member 10, the pushing portions 301 are each abutted and limited by the bottom cover 40, such that the fixing members 3 are each positioned relative to the main body member 51.

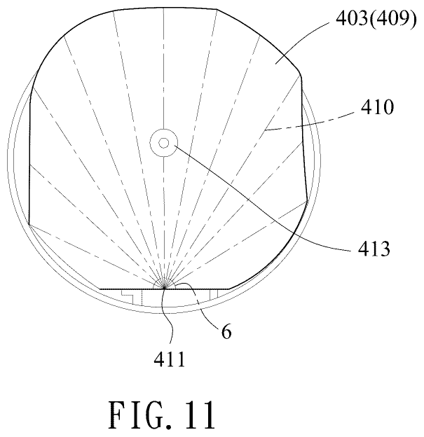

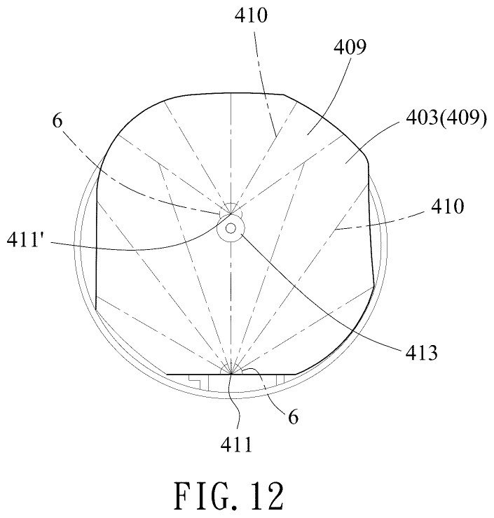

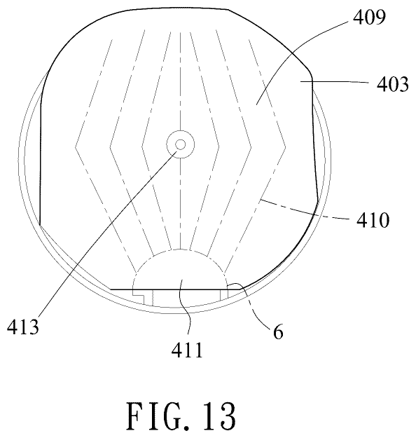

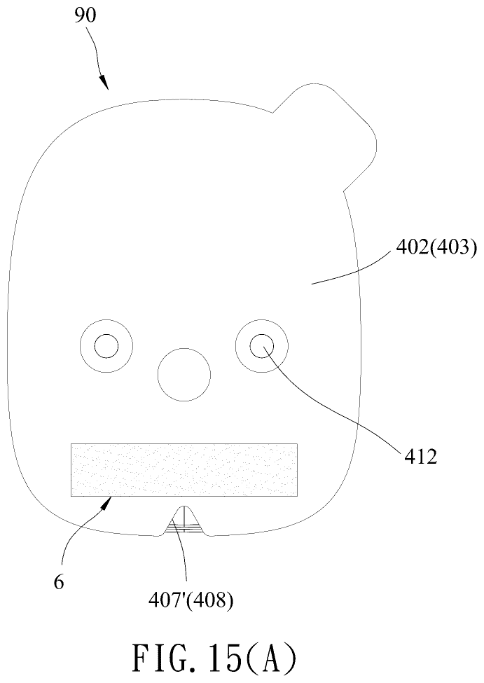

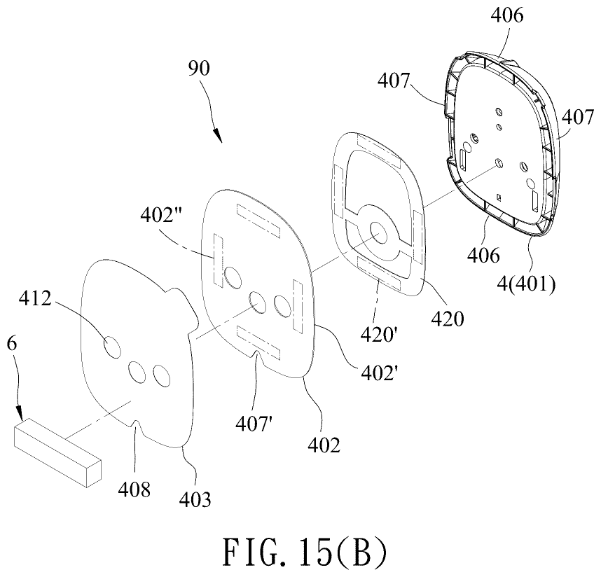

The bottom seat 4 is detachably limited to the main body unit 50. The bottom seat 4 has a main housing body 401 and an adhesive pad 402. The main housing body 401 has a buckling slot 400, a periphery 404, and two engaging slots 405 indented from a bottom of the periphery 404. The periphery 404 has a pair of first sides 406 and a pair of second sides 407 connected to the first sides 406, and a length of the first sides 406 is equal to or shorter than a length of the second sides 407. In one embodiment, the adhesive pad 402 is adhered to a release layer 403 that is detachably attached to the adhesive pad 402. The main housing body 401 is a hard material relative to the adhesive pad 402. The first engaging hooks 303 of the fixing members 3 may respectively engage the engaging slots 405. Referring to FIGS. 15 (A) and 15 (B), the adhesive pad 402 has a first notch 407′ corresponding to one of the first sides 406. The release layer 403 has a second notch 408 corresponding to the first notch 407′. The release layer 403 is divided into a number of blocks 409, and a cutting line 410 is formed between any two adjacent ones of the blocks 409. As a form shown in FIG. 11, the blocks 409 of the release layer 403 and the cutting lines 410 are arranged radially, and the cutting lines 410 have aggregated portions 411 that are mutually connected, and the aggregated portions 411 are disposed adjacent to a side edge of the release layer 403. As another form shown in FIG. 12, the release layer 403 has two groups of blocks 409 and two groups of cutting lines 410, and the two groups of blocks 409 and the two groups of cutting lines 410 are arranged radially. Each of the two groups of the cutting lines 410 has aggregated portions 411, 411′. The release layer 403 further has a through hole 413 provided for the sensor 502 to extend therethrough. One of the aggregated portions 411′ is disposed adjacent to the through hole 413. As one of forms shown in FIG. 13, the blocks 409 of the release layer 403 and the cutting lines 410 are arranged in a rhombus pattern, and the cutting lines 410 have aggregated portions 411 that are mutually connected.

The sensor component 5 is detachably limited to the bottom seat 4. The sensor component 5 includes a sensing base seat 501 and a sensor 502 connected to the sensing base seat 501 and extending into the implant needle 120. The sensing base seat 501 has two sleeve portions 503 provided for engaging the linked portions 781 and being concaved holes (only one of the sleeve portions 503 and one of the linked portions 781 are shown in FIG. 3 because of line of sight). The sensing base seat 501 is co-supported by the supporting portions 302 to remain positioned relative to the main body member 51. In one embodiment, the sleeve portions 503 that are convex (not shown), and the linked portions 781 that are concaved holes (not shown) are fitted to each other to prevent the sensing base seat 501 from rotation before implantation of the sensor.

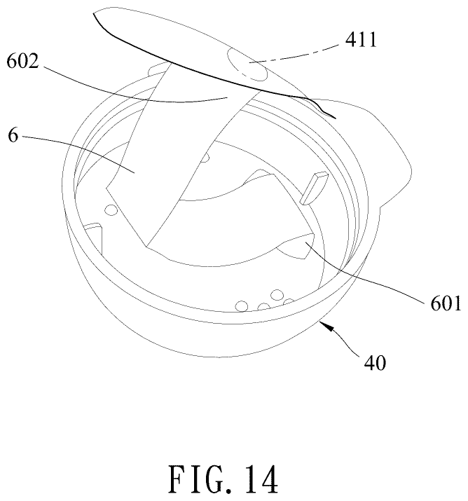

The peel-off element 6 is connected to the bottom seat 4 and the bottom cover 40. Furthermore, as shown in FIGS. 11 to 13, the peel-off element 6 is an adhesive, is connected to the bottom plate portion 41 and the aggregated portions 411 at a position located at an inner side of a corresponding one of the first sides 406, and is located at an inner side of the second notch 408 and adjacent to the second notch 408. As shown in FIG. 14, another form of the peel-off element 6 is a folded sheet and is folded into at least two folds, and the peel-off element 6 has a connecting end portion 601 connected to the bottom cover 40 and a tugging end portion 602 connected to the aggregated portions 411. As shown in FIGS. 15 (A) and 15 (B), another form of the peel-off element 6 may also be a foam material or other materials. In correspondence to such foam-employed peel-off element 6, the release layer 403 may not be provided with the blocks 409 and the cutting lines 410, but have a number of fine holes 412. When the bottom cover 40 is removed from the opening of the accommodating space 11, the release layer 403 may be teared off from the adhesive pad 402 through the peel-off element 6 that is connected to the bottom plate portion 41.

Referring to FIGS. 15 (A) and 15 (B), assembly and forming steps of the main housing body 401, the adhesive pad 402 and the release layer 403 of the bottom seat 4 are in the following.

(1) The bottom seat 4 performs an attaching step of a bonding layer 420, which is to attach the bonding layer 420 to a bottom surface of the main housing body 401. The bonding layer 420 is made of a polymeric material, such as thermoplastic polyurethane.

(2) A pre-hot press step is performed, which is to hot press from the bonding layer 420 in a direction toward the main housing body 401 so as to adhere the bonding layer 420 onto the main housing body 401. As shown in FIG. 15 (B), the bonding layer 420 is shown to have at least one hot press position 420′. In the pre-hot press step, the hot press is performed for 3 seconds to 10 seconds at a temperature of 75° C. to 85° C. and under a pressure of 3.5 kg/cm² to 4.5 kg/cm².

(3) An attachment step of an adhesive pad 402 is performed, which is to attach the adhesive pad 402 to the bonding layer 420, the adhesive pad 402 has an adhered surface 402′ that is attached to the release layer 403, and the adhesive pad 402 is shown to have a hot press position 402″.

(4) A hot press step is performed, which is to hot press from the adhesive pad 402 in a direction toward the main housing body 401 so as to bond the adhesive pad 402 onto the main housing body 401 through the bonding layer 420. In the hot press step, the hot press is performed for 10 seconds to 20 seconds at a temperature of 115° C. to 125° C. and under a pressure of 3.5 kg/cm² to 4.5 kg/cm².

(5) An attaching step of a peel-off element 6 is performed, which is to dispose the peel-off element 6 to at least correspond to a position among the hot press position 402″ of the adhesive pad 402, the hot press position 420′ of the bonding layer 420, and the bottom cover 40.

In order to further understand the functions produced, the technical means applied, and the expected effect achieved by cooperation of components of the present invention, they will be described again below, and it is believed that a more deep and specific understanding of the present invention can be obtained thereby.

As shown in FIGS. 1 and 2, 3, and 16 and 17, when the implant device of the present invention is entirely and completely assembled, the bottom cover 40 airtightly covers the housing member 10. At this time, the main body member 51 and the main body cover 52 are linked into one piece through mutually engagement of the engaging portions 513 and the connecting portions 521. Before a needle is stroke, a spacing is contained between the top cover 20 of the housing unit 1 and a top portion of the main body cover 52, and the snapped-on portions 76 of the implant seat 70 engage the buckling portions 514 of the main body member 51, such that the implant seat 70 is located at an upper position. The strike-limiting structure (A) composed between the implant seat 70 and the main body member 51 generates a safety lock, so the implant seat 70 is positioned relative to the main body member 51, and the first elastic member 81 is pre-compressed between the implant seat 70 and the main body cover 52 and stores a restoring resilient force. The limiting members 522 are inserted into the limiting grooves 752 and limit the buckling plates 751 to be unable to deviate radially. The needle withdrawal limiting structure (B) is utilized to enable the needle withdrawal seat 90 to generate a safety lock, and to position the needle withdrawal seat 90 relative to the implant seat 70. The second elastic member 82 is pre-compressed between the needle withdrawal seat 90 and the implant seat 70 and stores a restoring resilient force. The needle withdrawal seat 90 is at an un-strike position, and the sleeve portions 503 of the sensor component 5 are sleeved on the linked portions 781 such that the sensing base seat 501 is connected to the implant seat 70, so the implant needle 120 of the needle implant member 100 is hidden inside the main body member 51 and is concealed by the bottom seat 4, and the bottom seat 4 is positioned relative to the main body member 51. At the same time, the pushing portions 301 of the fixing members 3 are each abutted by the bottom cover 40, so as to position the fixing members 3 relative to the main body member 51. The sensing base seat 501 of the sensor component 5 is co-supported cooperatively by the supporting portions 302 so as to remain positioned relative to the main body member 51, and the first engaging hooks 303 engage respectively the engaging slots 405.

When the sensor 502 is to be implanted subcutaneously under a human body, the operations are described as follows.

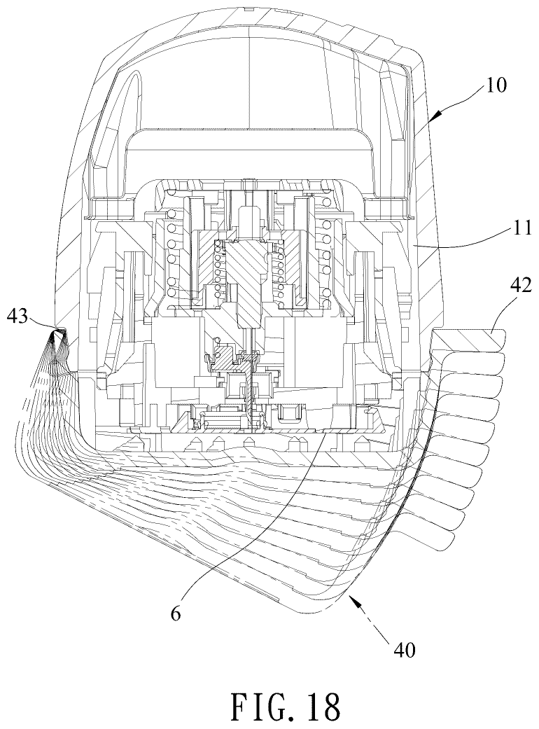

As shown in FIG. 18, an operator first detaches the bottom cover 40 from the housing member 10, and the bottom cover 40 has a force-exerting portion 42 thereon configured for bearing a force, and is formed with a supporting portion 43 at a corresponding side end of the force-exerting portion 42. A distance between the supporting portion 43 and the force-exerting portion 42 and the force form a force-exerting torque, and the force-exerting torque allows a user to rely on the force-exerting portion 42 to form a lateral regional opening between the bottom cover 40 and the housing member 10. When the bottom cover 40 is removed from the opening of the accommodating space 11, the release layer 403 may be peeled-off from the adhesive pad 402 through the effect of the peel-off element 6 that is connected to the bottom plate portion 41 of the bottom cover 40 and the aggregated portions 411 (referring to FIG. 11). After the release layer 403 is peeled off, the operator may adhere the adhesive pad 402 of the bottom seat 4 to a human body part to be implanted.

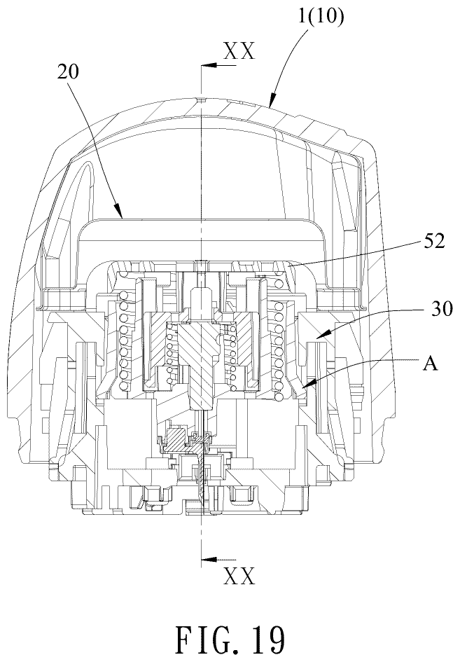

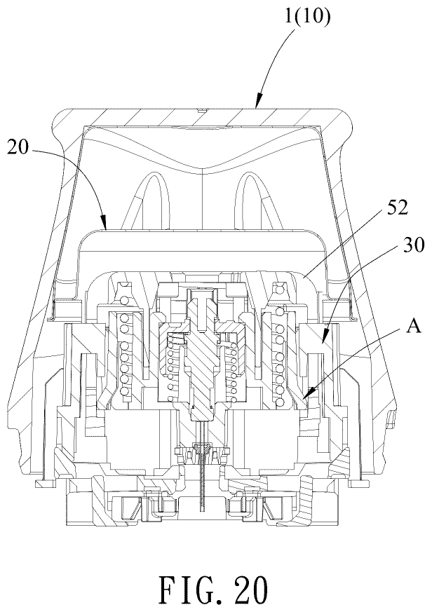

As shown in FIGS. 19 and 20, the operator exerts a force to press the housing member 10 of the housing unit 1, and the housing member 10 drives the top cover 20 and the lining member 30 to move toward the human body part, so the lining member 30 reaches a rushing down critical position where the strike-limiting structure (A) is to be released and the main body cover 52 is not moved yet.

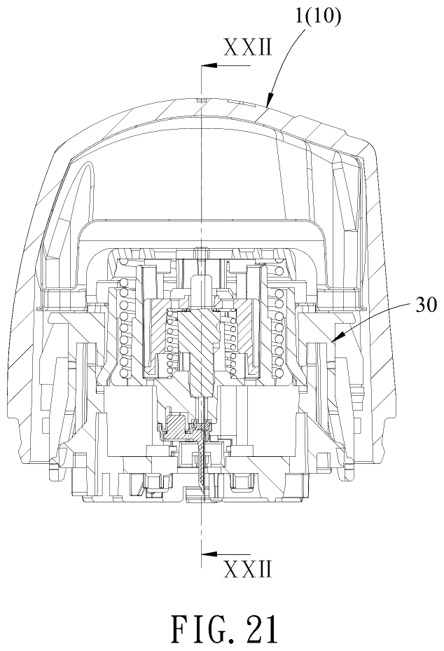

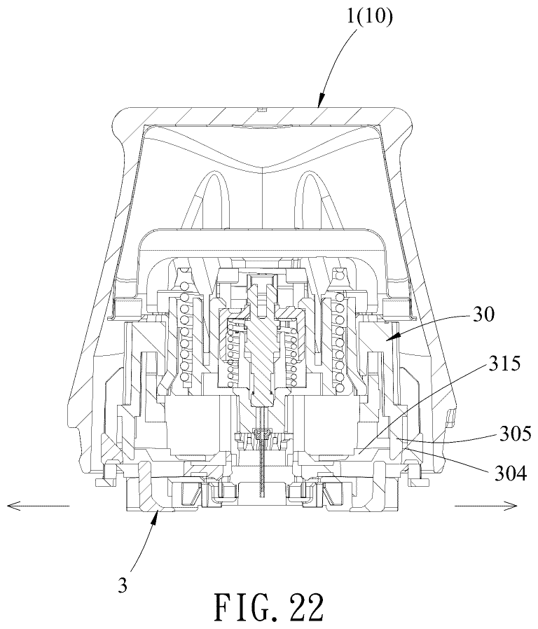

As shown in FIGS. 21 and 22, the operator continuously exerts a force to press the housing member 10 of the housing unit 1, and the pawls 35 of the lining member 30 generate a guiding and urging effect to the co-movable portions 304 of the fixing members 3 so as to move the fixing members 3 along the sliding grooves 515 outwardly. This is an action to unlock the fixing members 3 prior to striking, which in turn releases limitations thereof with respect to the sensor component 5 and the bottom seat 4.

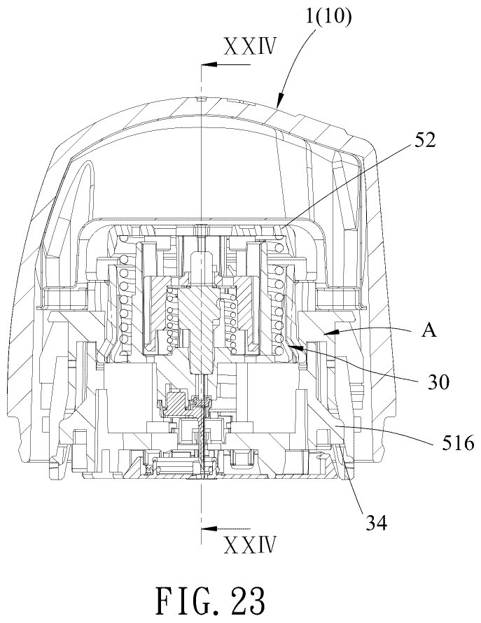

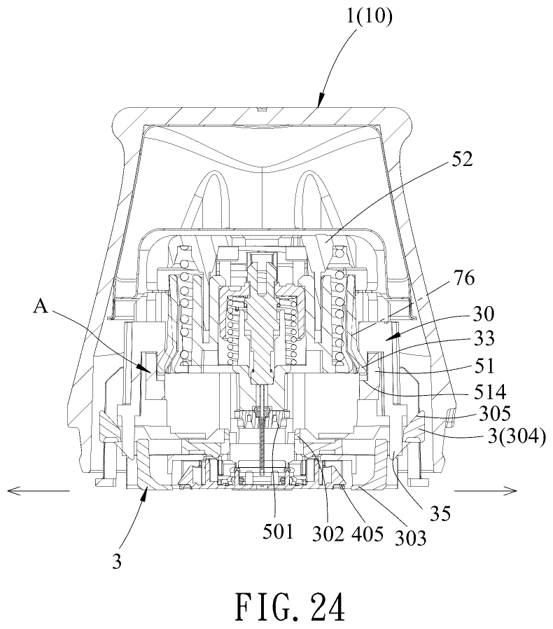

As shown in FIGS. 23 and 24, the operator continuously exerts a force to press the housing member 10 of the housing unit 1, so a distance between the lining member 30 and the main body cover 52 is reduced or disappears. At the same time, the driving portions 33 of the lining member 30 drive the snapped-on portions 76 of the implant seat 70 to disengage from the buckling portions 514 of the main body member 51. At the same time, the interlock portions 34 engage the corresponding limiting portions 516 and the lining member 30 engages and is positioned with respect to the main body member 51. Furthermore, the strike-limiting structure (A) is released, and after the snapped-on portions 76 of the implant seat 70 is disengaged the buckling portions 514 of the main body member 51, the main body member 51 releases limitation to the implant seat 70. When a force is exerted to press the housing unit 1, the pawls 35 of the lining member 30 drive the co-movable portions 304 of the fixing members 3, so the fixing members 3 are synchronizingly moved toward outside of the main body member 51 such that the supporting portions 302 detach from support to the sensing base seat 501, and that the first engaging hooks 303 detach respectively from the engaging slots 405, and a motion of the pawls 35 driving the fixing members 3 is smooth through an disposition of the guiding driving bevels 305.

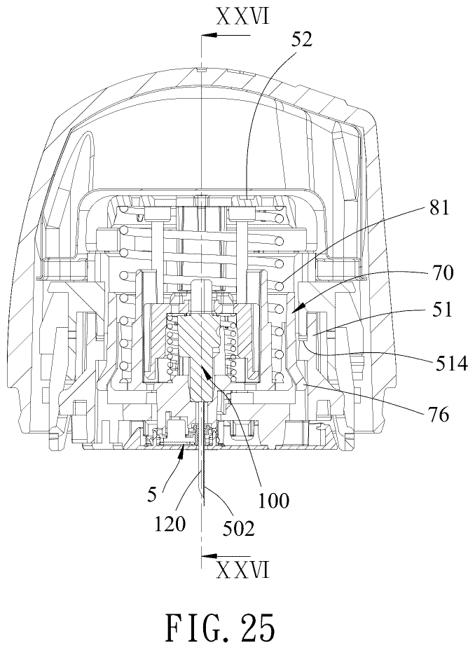

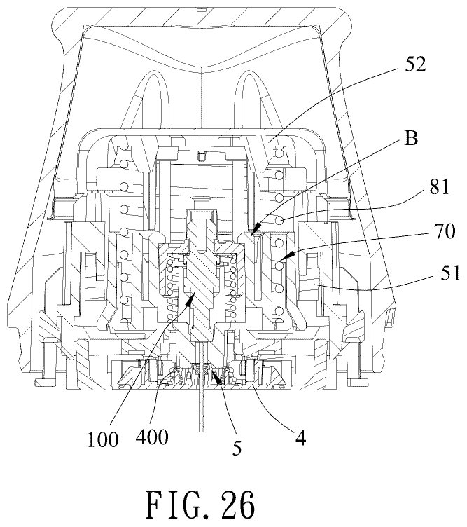

As shown in FIGS. 25 and 26, after the snapped-on portions 76 of the implant seat 70 are detached from the buckling portions 514 of the main body member 51, the main body member 51 releases limitation to the implant seat 70, so that the pre-compressed resilient force of the first elastic member 81 is released, and the resilient force of the first elastic member 81 is provided for moving the implant seat 70 in a needle implant direction away from the main body cover 52, and the implant seat 70 is located at a lower position, and the sensor component 5 is driven by the implant seat 70 and the needle implant member 100 to be at a striking position. The sensor 502 may be implanted under a skin of a user with the implant needle 120, and the sensing base seat 501 of the sensor component 5 engages the buckling slot 400 of the bottom seat 4.

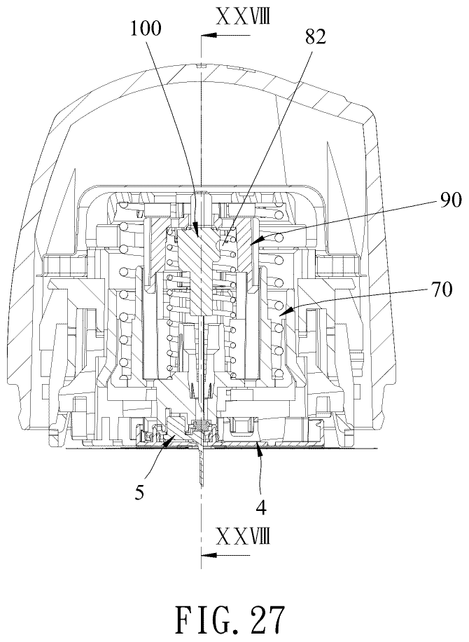

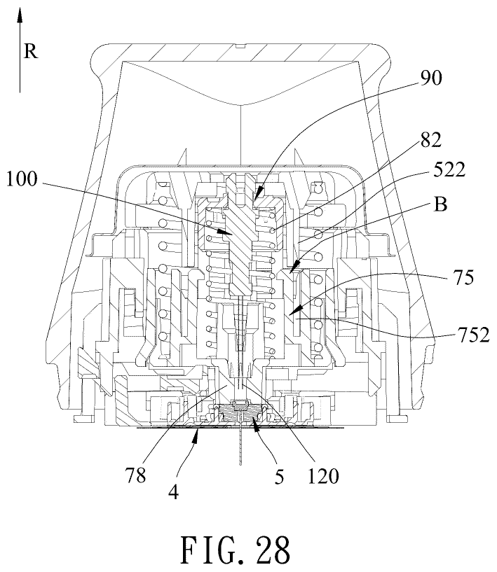

Then, as shown in FIGS. 27 and 28, the limiting members 522 of the main body cover 52 are separated from the limiting grooves 752 of the implant seat 70, so as to release locking of the limiting component 75 to the needle withdrawal seat 90, and the pre-compressed resilient force of the second elastic member 82 is also released, such that the needle withdrawal seat 90 drives the needle implant member 100 to move in a needle withdrawing direction (R) opposite to the needle implant direction, and the implant needle 120 is retracted into the implant seat 70. The positioning member 78 that is at the bottom of the implant seat 70 still presses the sensor component 5 against the bottom seat 4, which may prevent rebound of the sensor component 5 due to the kinetic energy of needle withdrawing.

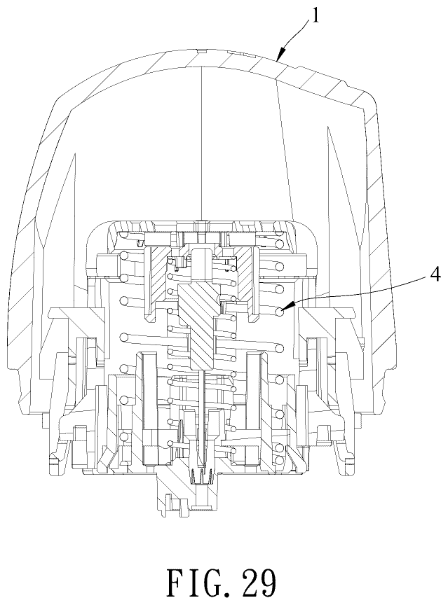

As shown in FIG. 29, the operator removes entirely the housing unit 1, the implant module 4, etc. relative to the human body surface and the bottom seat 4, so the bottom seat 4 and the sensor component 5 are retained on the human body surface (the bottom seat 4 and the sensor component 5 that are retained on the human body surface are not disclosed in FIG. 29).

The operator covers the bottom cover 40 that is originally detached on a bottom portion of the housing member 10, and the bottom cover 40 seals the bottom portion of the housing member 10.

The effects that may be generated by the present invention are summarized below.

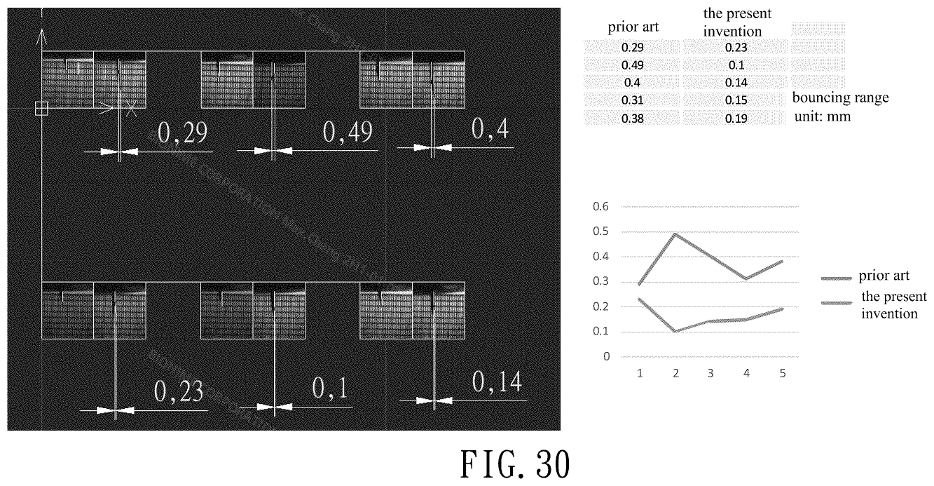

First, the guiding pins 61 of the guiding set 60 generate effect of a track. By utilizing the guiding set 60 that is connected to the main body unit 50 and that is located in the displacement space 53, the implant seat 70 and the needle withdrawal seat 90 are guided by the guiding set 60 to be displace smoothly. The achievable effects include easy control of production tolerance, increased production speed and yield rate, reduced needle bouncing, improved needle implantation stability, and more painless and sensationless needle implantation. Since a degree of the implant needle bouncing is significantly improved, arrangement of an auxiliary needle implant seat may be omitted in the implanter to facilitate stable needle implant as compared to the prior art, so components may be simplified. As shown in FIG. 30, experimental results of photographing through a high-speed camera for 5 times of test show that bouncing ranges of the prior art are 0.29 mm, 0.49 mm, 0.4 mm, 0.31 mm, and 0.38 mm, respectively. The bouncing ranges of the present invention that correspond thereto are 0.23 mm, 0.1 mm, 0.14 mm, 0.15 mm, and 0.19 mm, respectively. It is obvious that a degree of bouncing has been significantly decreased. By virtue of the design of adding the improved guiding set 60 in the implant device, in cooperation with needle implant actuating structures of the implant device, in a state where the implant device is pressed and is not released, the needle implant limiting structure (A) is released, the implant module 2 displaces downwardly by releasing the resilient force of the first elastic member 81 that is pre-compressed to complete automatic needle implantation, and the needle withdrawal limiting structure (B) is automatically released after the implant module 2 completes the automatic needle implantation. The implant module 2 displaces upwardly by releasing the resilient force of the second elastic member 82 that is pre-compressed to complete automatic needle withdrawal. Furthermore, during the process of the implant module 2 displacing downwardly to perform the automatic needle implantation, the sensor component 5 on the implant module 2 is positioned on the bottom seat 4, such that the duration for completing automatic needle implantation and automatic needle withdrawal is not greater than 100 milliseconds, or is not greater than 50 milliseconds, or is not greater than 8 milliseconds, 6 milliseconds, 4 milliseconds, or 2 milliseconds.

Second, an implant seat of the prior art cooperates with a trench to slide, and the trench has a design of a draft angle. In this way, the draft angle will cause a gap between the implant seat and a corresponding wall surface, resulting in bouncing of the implant needle. Moreover, as shown in FIG. 9, by virtue of the effect of each of the guiding tubes 74 that has the bottom segment 741, the top segment 742 and the hollow portion 743, contact areas among the guiding pins 61 and the guiding tubes 74 are smaller, so a draft gap that is caused in the manufacturing process may be overcome to likely achieve zero gap. Thus, during the implant seat 70 driving the needle withdrawal seat 90 to move downwardly, bouncing of the implant needle may be decreased significantly to decrease pain to thereby achieve a painless state.

Third, by utilizing the main body member 51 that is disposed with the first alignment mark 517, the main body cover 52 that is disposed with the second alignment mark 523, the needle withdrawal seat 90 that is disposed with the third alignment mark 92, the lining member 30 that is disposed with the fourth alignment mark 36, the implant seat 70 that is disposed with the fifth alignment mark 79, when manufacturing and assembly of relative components are performed, visual feature identification and automated orientation features are provided to generate an asymmetric foolproof effect.

Fourth, as shown in FIGS. 15 (A) and 15 (B), the adhesive pad 402 that is adjacent to the peel-off element 6 is provided with the first notch 407′, the release layer 403 is provided with the second notch 408, and the first notch 407′ and the second notch 408 may be used to permit gas to escape, to reduce a peel-off resistance, and to make it easier to tear. The design of a gap or a recess at an edge or a specific position of the release layer 403 provides a starting point for tearing, making tearing easier. Furthermore, microstructures such as fine holes 412 or microstructural embossed texture are introduced to a surface of the release layer 403, which facilitates in reducing adhesion. In addition, considering a direction of tearing, a tearing direction is designed to be along a weaker portion of the structure or its shape, making tearing easier to perform.

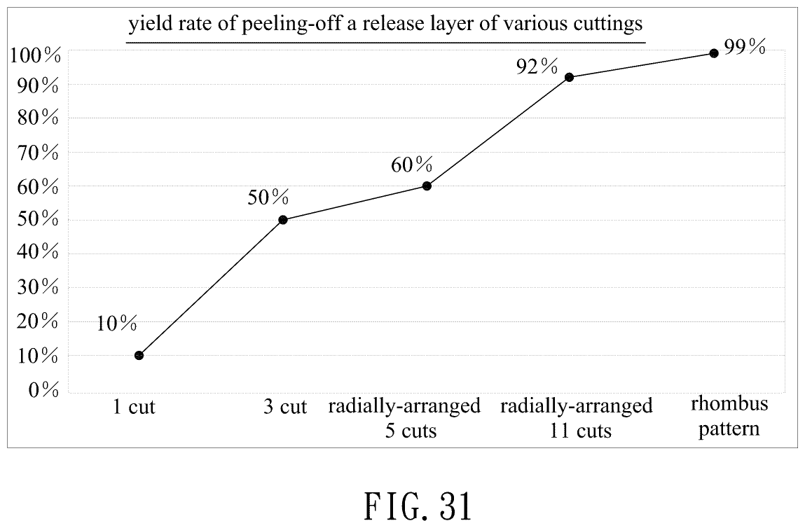

Fifth, as shown in FIGS. 11 and 12, and in cooperation with FIG. 31, the release layer 403 that is divided into the number of blocks 409, and the cutting line 410 that is formed between any two adjacent ones of the blocks 409 are utilized. Experimental results show that there is also a difference in yield rate of peeling-off the release layers obtained from different amounts of the cutting lines 410. For example, a yield rate of peeling-off the release layer is 10% when the cutting line 410 is 1 cut, a yield rate of peeling-off the release layer is 50% when the cutting lines 410 are 3 cuts, a yield rate of peeling-off the release layer is 60% when the cutting lines 410 radially arranged are 5 cuts, a yield rate of peeling-off the release layer is 92% when the cutting lines 410 radially arranged are 11 cuts (see FIG. 11), and a yield rate of peeling-off the release layer is 99% when the cutting lines 410 are arranged in a rhombus pattern (see FIG. 13). It is obvious that all of different forms of the cutting lines 410 of the present invention have a higher peel-off yield rate.

Sixth, in a state in which the implant device of the present invention is completely assembled and is not used yet, by filling the desiccant 7 into the chamber 21, an object of moisture resistance may be achieved so a detecting accuracy of the sensor component 5 is ensured.

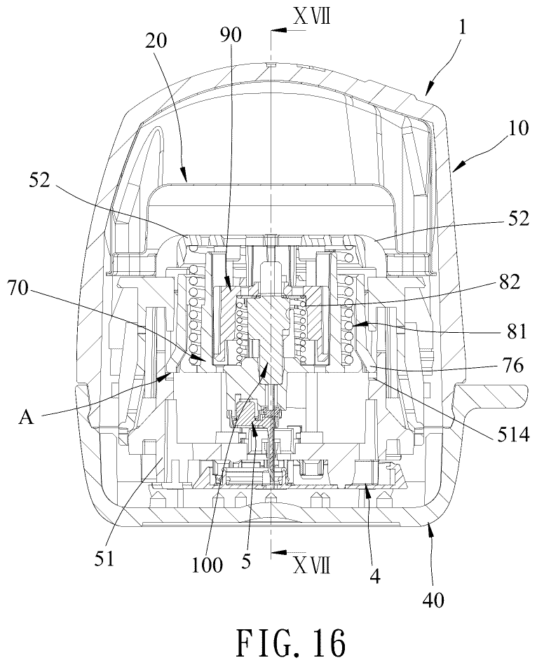

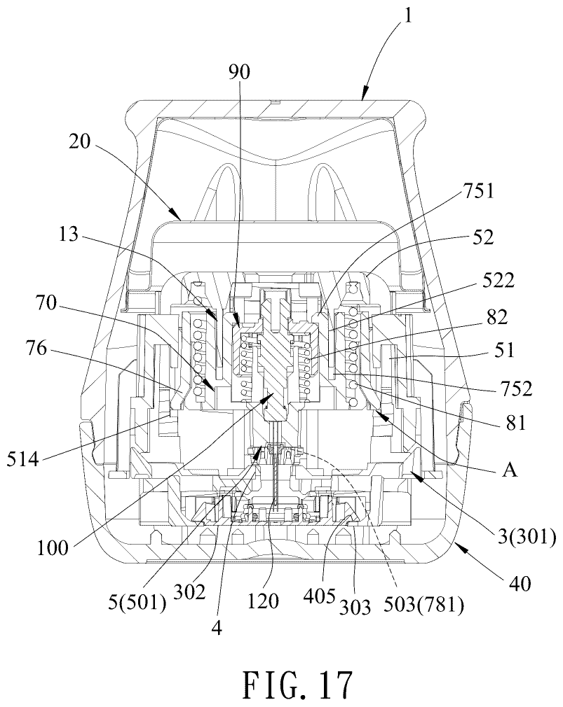

Seventh, as shown in FIGS. 16 and 17, when the implant device of the present invention is completely assembled and is not used yet, the pushing portions 301 of the fixing members 3 are each abutted by the bottom cover 40. By virtue of disposition of the fixing members 3, limitations to movement of the lining member 30 may be generated to prevent erroneous striking when the implant device accidentally falls or is operated by mistake, to thereby ensure the purpose of effective use. At the same time, the supporting portions 302 of the fixing members 3 generate a support effect to the sensing base seat 501 of the sensor, and position of the bottom seat 4 relative to the main body member 51 is generated by utilizing the engagement of the first engaging hooks 303 and the engaging slots 405.

It should be mentioned that the abovementioned embodiment of the present invention is described without disposition of an auxiliary needle implant seat. Certainly, an auxiliary needle implant seat (not shown) may be provided at a bottom portion of the positioning member 78. Referring to an auxiliary needle implant seat 38 disclosed in Taiwanese Invention Patent No. I723731 or U.S. Pat. No. 11,633,128, in addition to increasing components, an expected object of automatic needle implantation may also be achieved.

In summary, the implant device for a biosensor of the present invention may achieve easy control of production tolerance, increased production speed and yield rate, improved needle implantation stability, and more painless and sensationless needle implantation, which indeed achieves the object of the present invention.

The above is merely the embodiments of the present invention, and certainly the scope of the claims of the present invention cannot be limited thereby. Any simple equivalent variation and modification made according to the claims of the present invention and the patent specification should fall within the scope covered by the claims of the present invention.

1. An implant device for a biosensor, comprising:
a housing unit that has an accommodating space;
an implant module that is disposed in said accommodating space of said housing unit;
said implant module including:
a main body unit linked to said housing unit, said main body unit defining a displacement space;
a guiding set connected to said main body unit and located in said displacement space;
an implant seat detachably limited to said main body unit and guided by said guiding set to be displaced in said displacement space;
a first elastic member having one of ends that is positioned relative to said main body unit, and another one of said ends that resiliently abuts against said implant seat;
a needle withdrawal seat detachably limited to said implant seat and guided by said guiding set;
a second elastic member resiliently abutting between said implant seat and said needle withdrawal seat; and
a needle implant member;
a bottom seat that is detachably limited to said main body unit; and
a sensor component that is detachably limited to said bottom seat;
wherein said implant seat is displaced downwardly by releasing a resilient force of said first elastic member until a limiting relationship with said main body unit is released to thereby perform an automatic needle implantation; when the needle implantation is completed, the limiting relationship of said implant seat and said main body unit is released, and a limiting relationship of said implant seat and said needle withdrawal seat is released, so said needle withdrawal seat finishes an automatic needle withdrawal by releasing a resilient force of said second elastic member, and said implant seat and said needle withdrawal seat are guided by said guiding set to stably move.

2. The implant device for a biosensor as claimed in claim 1, wherein said main body unit of said implant module has a main body member and a main body cover detachably linked to said main body member, said guiding set has at least one guiding pin connected between said main body member and said main body cover, said implant seat has at least one first guiding hole provided for said guiding pin to slidably extend therethrough, and said needle withdrawal seat has at least one second guiding hole provided for said guiding pin to slidably extend therethrough.

3. The implant device for a biosensor as claimed in claim 2, wherein said main body member of said implant module has a bottom wall, an annular wall transversely connected to said bottom wall, a number of engaging portions disposed on said annular wall, and a first alignment mark, said main body cover having a number of connecting portions that mutually engage said engaging portions, and a second alignment mark that corresponds to said first alignment mark, said guiding set having more than two guiding pins connected to said bottom wall, an included angle being formed between any two adjacent ones of said guiding pins, the included angles being asymmetric.

4. The implant device for a biosensor as claimed in claim 3, wherein an amount of said second guiding holes of said needle withdrawal seat of said implant module corresponds to that of said guiding pins, an included angle being formed between any two adjacently disposed ones of said second guiding holes, the included angles being asymmetric, said needle withdrawal seat further having a third alignment mark that corresponds to said first alignment mark.

5. The implant device for a biosensor as claimed in claim 4, wherein said housing unit includes a lining member that has an inner peripheral surface, an outer peripheral surface opposite to said inner peripheral surface, and at least one driving portion disposed on said inner peripheral surface, said main body member having a buckling portion, said implant seat having at least one snapped-on portion that is driven by said driving portion and detachably engages said buckling portion, by abovementioned structural dispositions with respect to each other, a strike-limiting structure being composed between said implant seat and said main body unit.

6. The implant device for a biosensor as claimed in claim 5, wherein said implant seat and said main body cover of said implant module respectively have a limiting groove and a limiting member that are mutually limited, said implant seat further having a limiting component that is for limiting said needle withdrawal seat relative to said implant seat, by abovementioned structural dispositions with respect to each other, said implant seat, said main body cover and said needle withdrawal seat composing a needle withdrawal limiting structure.

7. The implant device for a biosensor as claimed in claim 2, wherein said implant seat of said implant module further has a plate member, an outer barrel member transversely connected to said plate member, an inner barrel member transversely connected to said plate member and located in said outer barrel member, and a number of guiding tubes connected to said inner barrel member, said first guiding holes being disposed respectively in said guiding tubes, each of said guiding tubes having a bottom segment that is connected to said plate portion, a top segment that is opposite to said bottom segment along an axial direction of a corresponding one of said guiding pins, and a hollow portion that is between said bottom segment and said top segment and communicates with a corresponding one of said first guiding holes.

8. The implant device for a biosensor as claimed in claim 7, wherein said implant seat of said implant module further has a positioning member, when said housing unit is not pressed downwardly, said sensor component is detachably mounted on said positioning member, and when said housing unit is pressed downwardly and the needle implantation is completed, said positioning member presses said sensor component against said bottom seat.

9. The implant device for a biosensor as claimed in claim 1, wherein said first elastic member and said second elastic member of said implant module may be pre-compressed springs.

10. The implant device for a biosensor as claimed in claim 5, wherein said housing unit further includes a housing member, a bottom cover engagable with said housing member in an airtight manner, and a top cover fixed in said housing member, said housing member and said top cover forming a chamber therebetween, a desiccant being disposed in said chamber.

11. The implant device for a biosensor as claimed in claim 1, wherein said housing unit further includes a bottom cover and a peel-off element connected to said bottom seat and said bottom cover, said bottom cover being used to detachably couple to an opening of said accommodating space of said housing member and including a bottom plate portion, said bottom seat including a main housing body, an adhesive pad fixed to said main housing body, and a release layer detachably attached to said adhesive pad, said peel-off element being connected to said bottom plate portion and said release layer, said release layer being peeled-off from said adhesive pad when said bottom cover is removed from said opening of said accommodating space.

12. The implant device for a biosensor as claimed in claim 11, wherein said release layer of said bottom seat is divided into a number of blocks, a cutting line being formed between any two adjacent ones of said blocks.

13. The implant device for a biosensor as claimed in claim 12, wherein said blocks and the cutting lines of said release layer of said bottom seat are radially arranged, the cutting lines having aggregated portions, said peel-off element being connected to said bottom plate portion and said aggregated portions.

14. The implant device for a biosensor as claimed in claim 13, wherein said blocks of said release layer of said bottom seat are arranged in a rhombus pattern, the cutting lines having aggregated portions, said peel-off element being connected to said bottom plate portion and said aggregated portions.

15. The implant device for a biosensor as claimed in claim 11, wherein said main housing body of said bottom seat has a periphery, said periphery having a pair of first sides and a pair of second sides connected to said first sides, said peel-off element being located at an inner side of a corresponding one of said first sides.

16. The implant device for a biosensor as claimed in claim 15, wherein said main housing body of said bottom seat is a hard material relative to said adhesive pad, and a disposition position of said peel-off element is relative to an inner edge of said bottom seat.

17. The implant device for a biosensor as claimed in claim 16, wherein said adhesive pad has a first notch corresponding to one of said first sides, said release layer having a second notch corresponding to said first notch, said peel-off element being located at an inner side of said second notch and adjacent to said second notch.

18. The implant device for a biosensor as claimed in claim 11, wherein said bottom seat further includes a bonding layer disposed between said main housing body and said adhesive pad, assembly and forming steps of said main housing body of said bottom seat, said adhesive pad, and said release layer being to first perform an attaching step of said bonding layer and to attach said bonding layer to a bottom surface of said main housing body, then to perform an attaching step of said adhesive pad, which is to attach said adhesive pad to said bonding layer, said adhesive pad having an adhered surface that is attached to said release layer, said adhesive pad being shown to have a hot press position, then to perform a hot press step, which is to perform hot press from said adhesive pad in a direction toward said main housing body so as to bond said adhesive pad to said main housing body through said bonding layer, and then to perform an attaching step of said peel-off element, which is to dispose said peel-off element to at least correspond to a position among the hot press position of said adhesive pad, a hot press position of said bonding layer, and said bottom cover.

19. The implant device for a biosensor as claimed in claim 18, wherein prior to performing the attaching step of said adhesive pad in the assembly and forming steps of said main housing body of said bottom seat, said adhesive pad, and said release layer, further includes a pre-hot press step, which is to perform hot press from said bonding layer in a direction toward said main housing body so as to adhere said bonding layer onto said main housing body.

20. An implant device for a biosensor, including:
a housing unit that includes a housing member;
an implant module that includes a main body unit, a guiding set, an implant seat, a first elastic member, a needle withdrawal seat, a second elastic member and a needle implant member;
a bottom seat; and
a sensor component;
wherein when said housing member is subjected to a force to activate needle implantation, said implant seat and said main body unit are released from a strike-limiting relationship, and said implant seat and said needle withdrawal seat are guided by said guiding set to move so said implant seat displaces downwardly to perform automatic needle implantation; after the needle implantation is completed, said implant seat and said needle withdrawal seat are released from a needle withdrawal limit relationship so said needle withdrawal seat displaces upwardly to complete automatic needle withdrawal, and said implant seat and said needle withdrawal seat are guided by said guiding set to stably move.

21. The implant device for a biosensor as claimed in claim 20, wherein said guiding set has at least one guiding pin connected between said main body member and said main body cover, said implant seat having at least one first guiding hole provided for slidably extension of said guiding pin therethrough, said needle withdrawal seat having at least one second guiding hole provided for slidably extension of said guiding pin therethrough.

22. The implant device for a biosensor as claimed in claim 21, wherein said guiding set is composed of at least more than two guiding pins, an included angle being formed between any two adjacently disposed ones of said guiding pins, the included angles being asymmetric.

23. The implant device for a biosensor as claimed in claim 22, wherein said implant seat has at least two guiding tubes disposed vertically in an axial direction, and each of said guiding tubes has a hollow portion in a horizontal direction.

24. The implant device for a biosensor as claimed in claim 20, wherein said housing unit includes a bottom cover, said bottom cover being detachably coupled to said housing member, said bottom seat being provided with an adhesive pad having a release layer that is attached thereto, a peel-off element being connected between said bottom cover and said release layer, when said bottom cover is removed, said peel-off element peeling off said release layer from said adhesive pad, a disposition position of said peel-off element being opposite to an inner edge of said bottom seat and being adjacent to a force-exerting portion of said bottom cover.
